(19) United States
(12) Patent Application Publication
(10) Pub. No.: US 2025/0188010 A1
(43) Pub. Date: Jun. 12, 2025

(54) RECYCLE CONTENT ORGANIC ACIDS FROM ALKOXY CARBONYLATION

(71) Applicant: Eastman Chemical Company, Kingsport, TN (US)

(72) Inventors: Kenneth Wayne Hampton, Jr., Kilgore, TX (US); Daryl Bitting, Longview, TX (US); Xianchun Wu, Longview, TX (US); David Eugene Slivensky, Tatum, TX (US)

(73) Assignee: Eastman Chemical Company, Kingsport, TN (US)

(21) Appl. No.: 18/845,508

(22) PCT Filed: Mar. 15, 2023
(86) PCT No.: PCT/US2023/064378

§ 371 Date: Sep. 10, 2024

Related U.S. Application Data
(60) Provisional application No. 63/269,486, filed on Mar. 17, 2022.

(51) Int. Cl. C07C 51/145; C01B 32/40

(52) U.S. Cl. CPC C07C 51/145; C01B 32/40

(57) ABSTRACT

Recycle content organic acids, such as recycle content propionic acid and recycle content butyric acid, are produced using a process and system that applies physical and/or credit-based recycle content from one or more feed materials to the organic acids produced from the feed materials. Thus, recycle content organic acids may be produced that contain physical recycle content and/or credit-based recycle content.

### BACKGROUND

Organic acids, such as propionic acid and butyric acid, are an important chemical used in a wide variety of applications. For example, propionic acid and butyric acid may be used to produce various chemical derivatives, including propionic anhydride and butyric anhydride, which may be used to produce mixed cellulose esters. Propionic acid is also used as a food preservative. Furthermore, butyric acid may be used to form various food and perfume additives.

The demand for recycled chemical products continues to grow, but there is no clear path to form recycle content organic acids, such as recycle content propionic acid and/or recycle content butyric acid, through mechanical recycling. Thus, there exists a need for a commercial process to produce recycle content organic acids.

### SUMMARY

In one aspect, the present technology concerns a process for producing a recycle content organic acid. Generally, the process comprises reacting ethylene and/or propylene with a C1-C6 alkanol, water, and carbon monoxide in the presence of a catalyst system to thereby produce a recycle content organic acid. Furthermore, at least a portion of the ethylene, propylene, carbon monoxide, or C1-C6 alkanol may comprise one or more of the following source materials: (i) a recycle content ethylene, (ii) a recycle content carbon monoxide, (iii) a recycle content C1-C6 alkanol, and/or (iv) a recycle content propylene.

In one aspect, the present technology concerns a process for producing a recycle content organic acid. Generally, the process comprises: (a) pyrolyzing a waste plastic to form a pyrolysis effluent comprising a first recycle content ethylene and/or a first recycle content propylene; (b) optionally cracking at least a portion of the pyrolysis effluent to form a second recycle content ethylene and/or a second recycle content propylene; (c) gasifying at least a portion of the pyrolysis effluent to form a recycle content syngas; (d) optionally forming a recycle content C1-C6 alkanol with at least a portion of the recycle content syngas; (e) optionally forming a recycle content carbon monoxide with at least a portion of the recycle content syngas; (f) optionally forming a third recycle content ethylene and/or a third recycle content propylene with at least a portion of the recycle content C1-C6 alkanol; and (g) reacting ethylene and/or propylene with a C1-C6 alkanol, water, and carbon monoxide in the presence of a catalyst system to thereby produce a recycle content organic acid. Furthermore, the recycle content organic acid comprises recycle content from one or more of the following source materials: (i) the waste plastic, (ii) the first recycle content ethylene, (iii) the first recycle content propylene, (iv) the second recycle content ethylene, (v) the second recycle content propylene, (vi) the recycle content syngas, (vii) the recycle content carbon monoxide, (viii) the recycle content C1-C6 alkanol, (ix) the third recycle content ethylene, and/or (x) the third recycle content propylene.

In one aspect, the present technology concerns a process for producing a recycle content organic acid. Generally, the process comprises: (a) treating a waste plastic to obtain, optionally through one or more intermediate processes, a recycle content ethylene, a recycle content propylene, a recycle content carbon monoxide, a recycle content C1-C6 alkanol or a combination thereof; and (b) reacting ethylene and/or propylene with a C1-C6 alkanol, water, and carbon monoxide in the presence of a catalyst system to thereby produce a recycle content organic acid. Furthermore, at least a portion of the ethylene, the propylene, or the carbon monoxide comprises the recycle content ethylene, the recycle content propylene, the recycle content C1-C6 alkanol, or the recycle content carbon monoxide, respectively.

### BRIEF DESCRIPTION OF THE DRAWINGS

FIG. 1 is a block flow diagram illustrating the main steps of a process and facility for making a recycle content organic acid (r-organic acid) that has physical recycle content from one or more source materials;

FIG. 2 is a block flow diagram illustrating the main steps of a process and facility for making a recycle content organic acid (r-organic acid), where the r-organic acid has credit-based recycle content from one or more source materials; and

FIG. 3 is a block flow diagram illustrating the main steps of a process and facility for making a recycle content organic acid (r-organic acid), where the r-organic acid has both physical and credit-based recycle content from one or more source materials.

### DETAILED DESCRIPTION

We have discovered new methods and systems for producing organic acids, such as propionic acid and butyric acid, having recycle content. More specifically, we have discovered a process and system for producing organic acids, where recycle content from waste materials, such as waste plastic, are applied to the organic acids in a manner that promotes the recycling of waste plastic and provides organic acids with substantial amounts of recycle content.

In general, organic acids, such as propionic acid and butyric acid, can be formed via a carbonylation reaction that involves reacting ethylene and/or propylene with a C1-C6 alkanol (e.g., methanol), water, and carbon monoxide in the presence of a catalyst system. During this reaction, at least a portion of the ethylene and/or propylene may be subjected to alkoxycarbonylation at elevated temperature and pressure followed by hydrolysis to thereby form propionic acid and/or butyric acid, respectively

The catalyst system used in the production of the organic acids can include the reaction product of: (a) a Group 8 to 10 transition metal compound, such as a palladium or ruthenium compound; and (b) an activating anion.

The recycle content organic acids produced in accordance with the present disclosure, such as the recycle content propionic acid and the recycle content butyric acid, can include recycle content from one or more source materials, including, for example, waste plastic, recycle content ethylene (r-ethylene), recycle content propylene (r-propylene), recycle content carbon monoxide (r-CO), recycle content syngas (r-syngas), and recycle content C1-C6 alkanol (e.g., recycle content methanol (r-methanol)). The recycle content in the recycle content organic acids, such as the recycle content propionic acid and the recycle content butyric acid, can be physical and may directly originate from at least one of the aforementioned streams, and/or the recycle content may be credit-based and applied to a target stream in the organic acid production process from one or more of these source streams.

The recycle content organic acids produced in accordance with the present disclosure, such as the recycle content propionic acid and/or the recycle content butyric acid, can have a total recycle content of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 weight percent, based on the total weight of the organic acid. Additionally, or in the alternative, the recycle content organic acids, such as the recycle content propionic acid and/or the recycle content butyric acid, can have a total recycle content of less than 99, less than 90, less than 80, less than 70, less than 60, less than 50, less than 40, less than 30, or less than 25 weight percent, based on the total weight of the organic acid. As used herein, the term “total recycle content” refers to the cumulative amount of physical recycle content and credit-based recycle content from all sources.

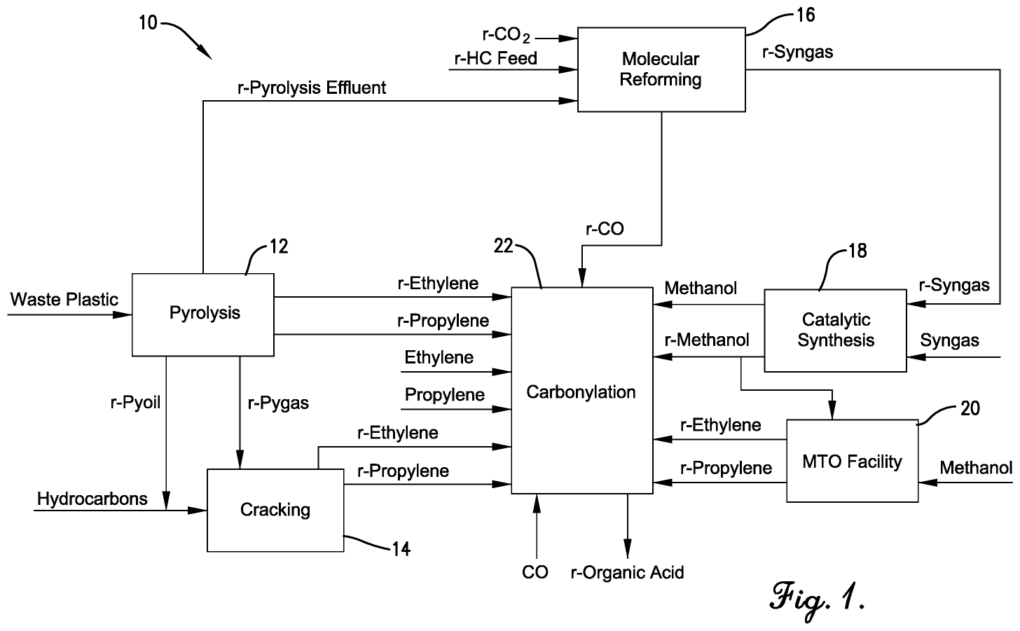

Turning now to FIG. 1, one embodiment of a facility 10 for forming organic acids with physical (direct) recycle content is provided.

The recycle content in the organic acids can originate from: (i) the pyrolysis 12 of waste plastic to form a recycle content pyrolysis effluent (r-pyrolysis effluent), which may comprise recycle content ethylene (r-ethylene), recycle content propylene (r-propylene), recycle content pyrolysis oil (r-pyoil), and/or recycle content pyrolysis gas (r-pygas); (ii) the cracking 14 of recycle content pyrolysis products (e.g., recycle content pyrolysis oil and/or recycle content pyrolysis gas) to thereby form r-ethylene and/or r-propylene; (iii) molecular reforming 16 of a recycle content hydrocarbon-containing feed (r-HC), a recycle content CO₂, an r-pyrolysis effluent, and a recycle content syngas (r-syngas) to form a recycle content carbon monoxide (r-CO); (iv) catalytic synthesis 18 of r-syngas to thereby form recycle content methanol (r-methanol); and/or (v) converting r-methanol in a Methanol-to-Olefins (MTO) facility 20 to thereby form r-ethylene and/or r-propylene.

### Pyrolysis Facility

As shown in FIG. 1, waste plastic can be pyrolyzed in a pyrolysis facility 12 to form a recycle content pyrolysis effluent (r-pyrolysis effluent), which may comprise recycle content ethylene (r-ethylene), recycle content propylene (r-propylene), recycle content pyrolysis oil (r-pyoil), and/or recycle content pyrolysis gas (r-pygas).

As used herein, the term “pyrolysis” refers to thermal decomposition of a feedstock of a biomass and/or a plastic material in solid or liquid form at elevated temperatures in an inert (i.e., substantially molecular oxygen free) atmosphere. A “pyrolysis facility” is a facility that includes all equipment, lines, and controls necessary to carry out pyrolysis of waste plastic and feedstocks derived therefrom.

In an embodiment or in combination with any embodiment mentioned herein, the waste plastic stream to the pyrolysis facility 12 may be a polyolefin-enriched stream of waste plastic. Furthermore, the plastic stream introduced into the pyrolysis reactor can be in the form of liquified plastic (e.g., liquified, melted, plasticized, depolymerized, or combinations thereof), plastic pellets or particulates, or a slurry thereof.

Pyrolysis is a process that involves the chemical and thermal decomposition of the introduced feed. Although all pyrolysis processes may be generally characterized by a reaction environment that is substantially free of oxygen, pyrolysis processes may be further defined, for example, by the pyrolysis reaction temperature within the reactor, the residence time in the pyrolysis reactor, the reactor type, the pressure within the pyrolysis reactor, and the presence or absence of pyrolysis catalysts.

In an embodiment or in combination with any embodiment mentioned herein, the pyrolysis reactor can be, for example, a film reactor, a screw extruder, a tubular reactor, a tank, a stirred tank reactor, a riser reactor, a fixed bed reactor, a fluidized bed reactor, a rotary kiln, a vacuum reactor, a microwave reactor, or an autoclave.

Furthermore, the temperature in the pyrolysis reactor can be adjusted so as to facilitate the production of certain end products. In an embodiment or in combination with any embodiment mentioned herein, the pyrolysis temperature in the pyrolysis reactor can range from 325 to 1,100° C., 350 to 900° C., 350 to 700° C., 350 to 550° C., 350 to 475° C., 425 to 1,100° C., 425 to 800° C., 500 to 1,100° C., 500 to 800° C., 600 to 1,100° C., 600 to 800° C., 650 to 1,000° C., 700 to 1,000° C., or 650 to 800° C. Generally, in certain embodiments, the pyrolysis temperature in the pyrolysis reactor can be greater than 650° C.

In an embodiment or in combination with any embodiment mentioned herein, the residence times of the feedstocks within the pyrolysis reactor can be at least 0.1, at least 0.2, at least 0.3, at least 0.5, at least 1, at least 1.2, at least 1.3, at least 2, at least 3, or at least 4 seconds. Alternatively, the residence times of the feedstocks within the pyrolysis reactor can be at least 1, at least 2, at least 3, at least 4, at least 5, at least 6, at least 7, at least 8, at least 9, at least 10, at least 20, at least 30, at least 45, at least 60, at least 75, or at least 90 minutes. Additionally, or alternatively, the residence times of the feedstocks within the pyrolysis reactor can be less than 6, less than 5, less than 4, less than 3, less than 2, less than 1, or less than 0.5 hours. Furthermore, the residence times of the feedstocks within the pyrolysis reactor can be less than 100, less than 90, less than 80, less than 70, less than 60, less than 50, less than 40, less than 30, less than 20, less than 10, less than 9, less than 8, less than 7, less than 6, less than 5, less than 4, less than 3, less than 2, or less than 1 seconds. More particularly, the residence times of the feedstocks within the pyrolysis reactor can range from 0.1 to 10 seconds, 0.5 to 10 seconds, 30 minutes to 4 hours, or 30 minutes to 3 hours, or 1 hour to 3 hours, or 1 hour to 2 hours.

In an embodiment or in combination with any embodiment mentioned herein, the pressure within the pyrolysis reactor can be maintained at atmospheric pressure or within the range of 0.1 to 100 bar, or 0.1 to 60 bar, or 0.1 to 30 bar, or 0.1 to 10 bar, 0.2 to 1.5 bar, or 0.3 to 1.1 bar. As used herein, the term “bar” refers to gauge pressure, unless otherwise noted.

In an embodiment or in combination with any embodiment mentioned herein, a pyrolysis catalyst may be present. The catalyst can be homogenous or heterogeneous and may include, for example, certain types of zeolites and other mesostructured catalysts.

In an embodiment or in combination with any embodiment mentioned herein, the pyrolysis reaction may not be catalyzed (e.g., carried out in the absence of a pyrolysis catalyst), but may include a non-catalytic, heat-retaining inert additive, such as sand, in the reactor in order to facilitate the heat transfer. Such catalyst-free pyrolysis processes may be referred to as “thermal pyrolysis.”

### Molecular Reforming Facility

As shown in FIG. 1, the organic acid production facility may also include a molecular reforming facility 16 for producing a recycle content carbon monoxide (r-CO) and a recycle content syngas (r-syngas) from at least a portion of the r-pyrolysis effluent, a recycle content CO₂ (r-CO₂), and/or a recycle content hydrocarbon feed (r-HC feed). The recycle content hydrocarbon feed can comprise waste plastics, recycle content hydrogen (r-H₂), recycle content solid hydrocarbons, recycle content liquid hydrocarbons, and/or recycle content gaseous hydrocarbons.

In an embodiment or in combination with any embodiment mentioned herein, the feed to the molecular reforming facility 16 can comprise a recycle content feed component (e.g., r-pyrolysis effluent, r-CO₂, and/or r-HC feed) and a non-recycle content feed component (e.g., coal, a liquid hydrocarbon, and/or a gaseous hydrocarbon).

In an embodiment or in combination with any embodiment mentioned herein, the molecular reforming is partial oxidation gasification that is fed with coal and waste plastic. In yet other embodiments, the molecular reforming is plasma gasification of a predominately waste plastic feed. In yet even other embodiments, the molecular reforming is partial oxidation gasification fed with a non-recycle content liquid or gaseous hydrocarbon and a recycle content pyrolysis oil produced from the pyrolysis of waste plastic.

In an embodiment or in combination with any embodiment mentioned herein, the molecular reforming can include catalytic reforming, while in other embodiments, the carbon reforming can include steam reforming.

Exemplary molecular reforming facilities can include a partial oxidation (POX) gasification facility or a steam reforming facility. In an embodiment or in combination with any embodiment mentioned herein, the molecular reforming facility 16 may comprise a POX gasifier.

In an embodiment or in combination with any embodiment mentioned herein, the molecular reforming facility 16 comprises a POX gasification facility. The feed to POX gasification can include solids, liquids, and/or gases.

In the POX gasification facility, at least a portion of the r-pyrolysis effluent, a recycle content CO₂ (r-CO₂), and/or a recycle content hydrocarbon feed (r-HC feed) may be converted to r-syngas and/or r-CO in the presence of a sub-stoichiometric amount of oxygen.

The POX gasification facility includes at least one POX gasification reactor. The POX gasification unit may comprise a gas-fed reactor (or gasifier). In an embodiment or in combination with any embodiment mentioned herein, the POX gasification facility may perform gas-fed POX gasification. As used herein, “gas-fed POX gasification” refers to a POX gasification process where the feed to the process comprises predominately (by weight) components that are gaseous at 25° C. and 1 atm.

Gas-fed POX gasification processes can be co-fed with lesser amounts of other components having a different phase at 25° C. and 1 atm. Thus, gas-fed POX gasifiers can be co-fed with liquids and/or solids, but only in amounts that are less (by weight) than the amount of gases fed to the gas-phase POX gasifier.

In an embodiment or in combination with any embodiment mentioned herein, the total feed to a gas-fed POX gasifier can comprise at least 60, at least 70, at least 80, at least 90, or at least 95 weight percent of components that are gaseous at 25° C. and 1 atm.

In an embodiment or in combination with any embodiment mentioned herein, the gasification zone, and optionally all reaction zones in the gasifier/gasification reactor, may be operated at a temperature of at least 1000° C., at least 1100° C., at least 1200° C., at least 1250° C., or at least 1300° C. and/or not more than 2500° C., not more than 2000° C., not more than 1800° C., or not more than 1600° C. The reaction temperature may be autogenous. Advantageously, the gasifier operating in steady state mode may be at an autogenous temperature and does not require application of external energy sources to heat the gasification zone.

In an embodiment or in combination with any embodiment mentioned herein, the gasifier may be operated at a pressure within the gasification zone (or combustion chamber) of at least 200 psig (1.38 MPa), at least 300 psig (2.06 MPa), at least 350 psig (2.41 MPa), at least 400 psig (2.76 MPa), at least 420 psig (2.89 MPa), at least 450 psig (3.10 MPa), at least 475 psig (3.27 MPa), at least 500 psig (3.44 MPa), at least 550 psig (3.79 MPa), at least 600 psig (4.13 MPa), at least 650 psig (4.48 MPa), at least 700 psig (4.82 MPa), at least 750 psig (5.17 MPa), at least 800 psig (5.51 MPa), at least 900 psig (6.2 MPa), at least 1000 psig (6.89 MPa), at least 1100 psig (7.58 MPa), or at least 1200 psig (8.2 MPa). Additionally or alternatively, the gasifier may be operated at a pressure within the gasification zone (or combustion chamber) of not more than 1300 psig (8.96 MPa), not more than 1250 psig (8.61 MPa), not more than 1200 psig (8.27 MPa), not more than 1150 psig (7.92 MPa), not more than 1100 psig (7.58 MPa), not more than 1050 psig (7.23 MPa), not more than 1000 psig (6.89 MPa), not more than 900 psig (6.2 MPa), not more than 800 psig (5.51 MPa), or not more than 750 psig (5.17 MPa).

In an embodiment or in combination with any embodiment mentioned herein, the gasifier is a non-slagging gasifier or operated under conditions not to form a slag.

### Methanol Catalytic Synthesis Facility

Turning again to FIG. 1, at least a portion or all of the r-syngas from the molecular reforming facility 16 can be introduced into a methanol catalytic synthesis facility 18 so as to produce recycle content methanol (r-methanol). This catalytic synthesis process can occur in the presence of a catalyst, at a temperature in the range of 150 to 400° C., and pressure in the range of 600 to 1,700 psig.

The reaction can be a gas-phase reaction or a liquid-phase reaction. Furthermore, the catalyst can comprise copper, zinc oxide, alumina, and/or magnesia.

In an embodiment or in combination with any embodiment mentioned herein, the feed to the methanol catalytic synthesis facility 18 can comprise r-syngas and non-recycle content syngas.

As used herein, the term “methanol catalytic synthesis facility” refers to a facility that includes all equipment, lines, and controls necessary to carry out the methanol catalytic synthesis process.

### MTO Facility

Turning once again to FIG. 1, at least a portion or all of the r-methanol from the methanol catalytic synthesis facility can be routed to a Methanol-to-Olefins (MTO) facility 20. While in the MTO facility, at least a portion of the r-methanol can be converted into additional r-ethylene and/or r-propylene streams.

Generally, the MTO process involves two reaction steps: (1) at least a portion of the methanol is converted to dimethyl ether and water and (2) at least a portion of the dimethyl ether is converted into ethylene and propylene. The ratios of ethylene and propylene in the product can vary depending on reaction conditions and catalyst type. Generally, the zeolite can be a zeolite type catalyst, such as ZSM-5 and the reactions can occur in a fluidized bed reactor at a temperature in the range of 300 to 500° C. and at or near atmospheric pressure.

Exemplary MTO facilities and processes that may be utilized include the UOP/Hydro MTO process, the Lurgi MTP Process, and the Olefin Cracking Process (OCP) by Total.

### Cracking Facility

Turning again to FIG. 1, all or a portion of the r-pygas and/or r-pyoil can be introduced into a cracking facility 14, where it can be used to produce a recycle content olefin, such as additional r-ethylene and/or r-propylene. The feed to the cracking facility 14 can include only r-pyoil and/or r-pygas, or it can also include a non-recycle content hydrocarbon, such as naphtha (e.g., C5 to C22) or lighter hydrocarbon components (e.g., C2 to C5).

As used herein, the term “cracking” refers to breaking down complex organic molecules into simpler molecules by the breaking of carbon-carbon bonds. A “cracking facility” is a facility that includes all equipment, lines, and controls necessary to carry out cracking of a feedstock derived from waste plastic. A cracking facility 14 can include one or more cracker furnaces, a quench system for cooling the cracked products, a compression system, and a downstream separation zone including equipment used to process the effluent of the cracker furnace(s). As used herein, the terms “cracker” and “cracking” are used interchangeably. In certain embodiments, the cracking facility 14 may comprise at least one steam cracker.

In an embodiment or in combination with any embodiment mentioned herein, the cracking facility 14 may comprise at least one fluidized catalytic cracker.

The cracker facility 14 may include a cracker furnace and a separation zone downstream of the cracker furnace for separating the furnace effluent into various end products, such as a recycle content hydrocarbons (e.g., r-propylene and r-ethylene).

In an embodiment or in combination with any embodiment mentioned herein, at least a portion of the r-pyoil stream and/or the r-pygas stream can be sent to the cracking facility 14. The r-pyoil stream may be introduced into an inlet of the cracker furnace, while the r-pygas stream can be introduced into a location upstream or downstream of the furnace. When used, the r-pyoil stream and/or r-pygas stream may optionally be combined with a stream of hydrocarbon feed to form the feed stream to the cracking facility.

In an embodiment or in combination with any embodiment mentioned herein, the hydrocarbon feed stream can comprise at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, or at least 95 weight percent of pyrolysis gas, pyrolysis oil, or pyrolysis gas and pyrolysis oil combined, based on the total weight of the stream. Alternatively, or in addition, the hydrocarbon feed stream can comprise not more than 95, not more than 90, not more than 85, not more than 80, not more than 75, not more than 70, not more than 65, not more than 60, not more than 55, not more than 50, not more than 45, not more than 40, not more than 35, not more than 30, not more than 25, or not more than 20 weight percent of pyrolysis gas, pyrolysis oil, or a combination of pyrolysis gas and pyrolysis oil, based on the total weight of the stream, or it can include these components in an amount in the range of from 1 to 95 weight percent, 5 to 90 weight percent, or 10 to 85 percent, based on the total weight of the stream.

In an embodiment or in combination with any embodiment mentioned herein, the cracker facility 14 may comprise a single cracking furnace, or it can have at least 2, or at least 3, or at least 4, or at least 5, or at least 6, or at least 7, or at least 8 or more cracking furnaces operated in parallel. Any one or each furnace(s) may be gas cracker, or a liquid cracker, or a split furnace.

The cracker feed stream, along with the pyrolysis oil stream and/or pyrolysis gas, may pass through the cracking furnace, wherein the hydrocarbon components therein are thermally cracked to form lighter hydrocarbons, including olefins such as ethylene, propylene, and/or butadiene. The residence time of the cracker stream in the furnace can be in the range of from 0.15 to 2 seconds, 0.20 to 1.75 seconds, or 0.25 to 1.5 seconds.

The temperature of the cracked olefin-containing effluent withdrawn from the furnace outlet can be in the range of from 730 to 900° C., 750 to 875° C., or 750 to 850° C.

Upon exiting the cracker furnace outlet, the olefin-containing effluent stream may be cooled rapidly (e.g., quenched) in the quench system (not shown) in order to prevent production of large amounts of undesirable by-products and to minimize fouling in downstream equipment. In an embodiment or in combination with any embodiment mentioned herein, the temperature of the olefin-containing effluent from the furnace can be reduced by 35 to 485° C., 35 to 375° C., or 90 to 550° C. to a temperature of 500 to 760° C. during the quench or cooling step.

The resulting cooled effluent stream can be then separated in a vapor-liquid separator, and the vapor can be compressed in a gas compressor having, for example, between 1 and 5 compression stages with optional inter-stage cooling and liquid removal. The pressure of the gas stream at the outlet of the first set of compression stages is in the range of from 7 to 20 bar gauge (barg), 8.5 to 18 barg, or 9.5 to 14 barg.

The compressed olefin-containing stream may then be further compressed in another compressor, optionally with inter-stage cooling and liquid separation. The resulting compressed stream, which has a pressure in the range of 20 to 50 barg, 25 to 45 barg, or 30 to 40 barg. The resulting stream may then be passed to a fractionation section in the cracking facility, wherein the olefins and other components may be separated into various high-purity product or intermediate streams, including an r-ethylene stream and a r-propylene stream.

In an embodiment or in combination with any embodiment mentioned herein, all or a portion of the pyrolysis gas may be introduced prior to and/or after one or more stages of the second compressor.

### Carbonylation Facility

As shown in FIG. 1, all or a portion of: (i) the r-ethylene and/or r-propylene from the pyrolysis facility 12, the cracking facility 14, and/or the MTO facility 20; (ii) the r-CO from the molecular reforming facility 16; and/or (iii) the r-methanol from the catalytic synthesis facility 18 can be sent and used in the carbonylation facility 22 described herein. Additionally, or in the alternative, at least a portion of the feed to the carbonylation reactor can include non-recycle content ethylene, non-recycle content propylene, non-recycle content CO, and/or non-recycle content methanol.

In an embodiment or in combination with any embodiment mentioned herein, the feed to the carbonylation facility 22 can comprise at least at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 weight percent recycle content materials, such as r-ethylene, r-propylene, r-CO, r-methanol, or combinations thereof.

Additionally, or in the alternative, the feed to the carbonylation facility 22 can comprise less than 99, less than 90, less than 80, less than 70, less than 60, less than 50, less than 40, less than 30, or less than 25 weight percent recycle content materials, such as r-ethylene, r-propylene, r-CO, r-methanol, or combinations thereof.

In an embodiment or in combination with any embodiment mentioned herein, all or at least a portion of the r-methanol, the r-CO, the r-ethylene, and/or the r-propylene streams depicted in FIG. 1 can be subjected to carbonylation to form the recycle content organic acids, such as recycle content propionic acid and/or recycle content butyric acid.

Generally, the process comprises reacting ethylene and/or propylene with a C1-C6 alkanol (such as methanol), water, and carbon monoxide in the presence of a catalyst system to thereby produce a recycle content organic acid. Furthermore, at least a portion of the ethylene, propylene, carbon monoxide, or C1-C6 alkanol may comprise one or more of the following source materials: (i) the recycle content ethylene, (ii) the recycle content carbon monoxide, (iii) the recycle content methanol, and/or (iv) the recycle content propylene.

The carbonylation catalyst system used in the production of the organic acids can include the reaction product of: (a) a Group 8 to 10 transition metal compound, such as a palladium or ruthenium compound; and (b) an activating anion.

The carbonylation reaction may be conducted at temperatures from 50 to 200° C., or between 75 and 165° C. In an embodiment or in combination with any embodiment mentioned herein, the carbonylation reaction is conducted at a pressure of least 1,375 kPa. In an embodiment or in combination with any embodiment mentioned herein, the reaction is conducted at pressures from 1,375 kPa to 4,100 kPa, or from 2,000 kPa to 3,450 kPa.

In an embodiment or in combination with any embodiment mentioned herein, the ethylene used in the carbonylation facility 22 can include at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent recycle content. Additionally, or in the alternative, the ethylene used in the carbonylation facility can include less than 99, less than 90, less than 80, less than 70, less than 60, less than 50, less than 40, less than 30, or less than 25 percent recycle content. In certain embodiments, all of the ethylene used in the carbonylation facility can include recycle content. This recycle content may be derived from physical recycle content via the pyrolysis, cracking, molecular reforming, catalytic synthesis, and/or MTO processes described herein.

In an embodiment or in combination with any embodiment mentioned herein, the propylene used in the carbonylation facility 22 can include at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent recycle content. Additionally, or in the alternative, the propylene used in the carbonylation facility 22 can include less than 99, less than 90, less than 80, less than 70, less than 60, less than 50, less than 40, less than 30, or less than 25 percent recycle content. In certain embodiments, all of the propylene used in the carbonylation facility 22 can include recycle content. This recycle content may be derived from physical recycle content via the pyrolysis, cracking, molecular reforming, catalytic synthesis, and/or MTO processes described herein.

In an embodiment or in combination with any embodiment mentioned herein, the CO used in the carbonylation facility 22 can include at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent recycle content. Additionally, or in the alternative, the CO used in the carbonylation facility 22 can include less than 99, less than 90, less than 80, less than 70, less than 60, less than 50, less than 40, less than 30, or less than 25 percent recycle content. In certain embodiments, all of the CO used in the carbonylation facility 22 can include recycle content. This recycle content may be derived from physical recycle content via the waste plastics, pyrolysis, cracking, molecular reforming, catalytic synthesis, and/or MTO processes described herein.

In an embodiment or in combination with any embodiment mentioned herein, the C1-C6 alkanol, such as methanol, used in the carbonylation facility can include at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent recycle content. Additionally, or in the alternative, the C1-C6 alkanol, such as methanol, used in the carbonylation facility 22 can include less than 99, less than 90, less than 80, less than 70, less than 60, less than 50, less than 40, less than 30, or less than 25 percent recycle content. In certain embodiments, all of the C1-C6 alkanol, such as methanol, used in the carbonylation facility can include recycle content. This recycle content may be derived from physical recycle content via the waste plastics, pyrolysis, cracking, molecular reforming, catalytic synthesis, and/or MTO processes described herein.

The carbonylation reaction can be conducted in a solvent that may at least partially dissolve the homogeneous catalyst system, the C1-C6 alkanol (e.g., methanol), and the gaseous components. The solvent can be any non-reacting material, such as a hydrocarbon, ester, ether, or amine. Alternatively, the solvent may be a compound which takes part in the reaction, either as a reactant or a product, to minimize the number of different compounds present in the liquid phase to facilitate separation of the mixture. In an embodiment or in combination with any embodiment mentioned herein, ethylene and/or propylene are carbonylated in the presence of carbon monoxide and methanol to form methyl propionate, utilizing methyl propionate as a solvent. Additionally, or in the alternative, the solvent may be N-methyl-2-pyrrolidinone.

The hydrolysis portion of the carbonylation reaction may be carried out by the water added to the reactor. The amount of water can vary but should be enough to exceed about 1 molar equivalent of the methanol added to the reactor and thus exceed about 1 molar equivalent of the theoretical methyl propionate that may be formed. The hydrolysis may be carried out in the same reactor at the same temperature and pressure as the carbonylation reaction. Thus, the carbonylation reaction can be referred to as a “one-pot” synthesis of organic acid.

The resulting r-organic acid can have a recycle content of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent derived from physical recycle content. Additionally, or in the alternative, the resulting r-organic acid can include less than 99, less than 90, less than 80, less than 70, less than 60, less than 50, less than 40, less than 30, or less than 25 percent recycle content derived from physical recycle content. In certain embodiments, all of the recycle content in the r-organic acid may be derived from physical recycle content.

As noted above, the r-organic acid produced in the carbonylation facility may be r-propionic acid and/or r-butyric acid. Thus, the resulting r-propionic acid and/or r-butyric acid produced in the carbonylation facility may have a recycle content of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent derived from physical recycle content. Additionally, or in the alternative, the r-propionic acid and/or the r-butyric acid can include less than 99, less than 90, less than 80, less than 70, less than 60, less than 50, less than 40, less than 30, or less than 25 percent recycle content derived from physical recycle content. In certain embodiments, all of the recycle content in the r-propionic acid and/or r-butyric acid may be derived from physical recycle content.

The amount of physical recycle content in the r-organic acids can be determined by tracing the amount of recycled material along a chemical pathway starting with waste plastic and ending with the organic acid. The chemical pathway includes all chemical reactions and other processing steps (e.g., separations) between the starting material (e.g., waste plastic) and the organic acid. In FIG. 1, the chemical pathway includes pyrolysis, cracking, molecular reforming, catalytic synthesis, the MTO process, and carbonylation.

In an embodiment or in combination with any embodiment mentioned herein, a conversion factor can be associated with each step along the chemical pathway. The conversion factors account for the amount of the recycle content diverted or lost at each step along the chemical pathway. For example, the conversion factors can account for the conversion, yield, and/or selectivity of the chemical reactions along the chemical pathway.

The amount of recycle content applied to the r-organic acid, or any of the recycle content streams disclosed herein, can be determined using one of variety of methods for quantifying, tracking, and allocating recycle content among various materials in various processes. One suitable method, known as “mass balance,” quantifies, tracks, and allocates recycle content based on the mass of the recycle content in the process. In certain embodiments, the method of quantifying, tracking, and allocating recycle content is overseen by a certification entity that confirms the accuracy of the method and provides certification for the application of recycle content to the r-organic acid.

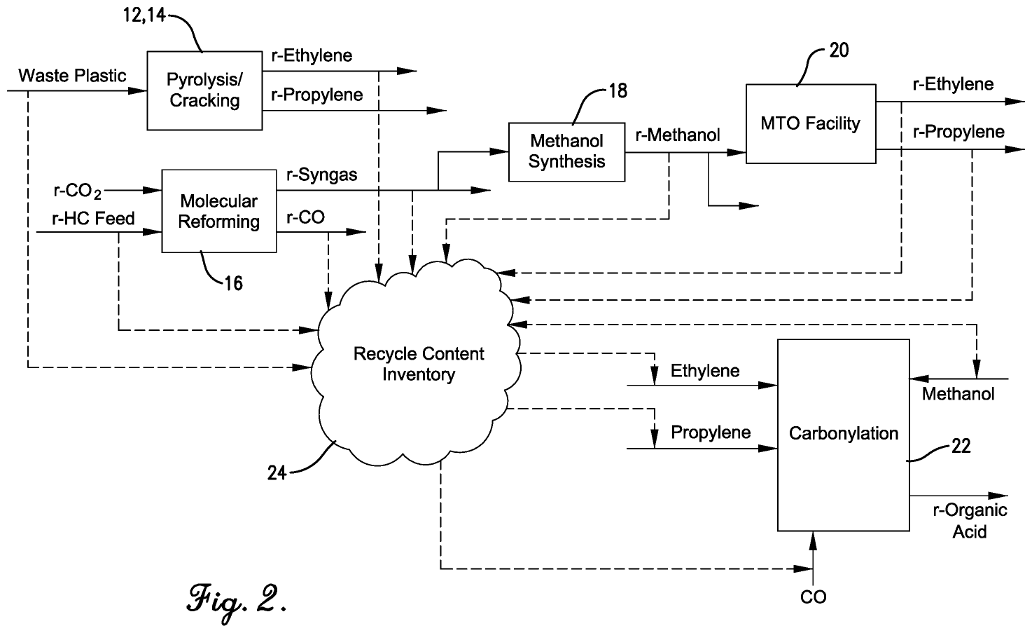

Turning now to FIG. 2, an embodiment where the r-organic acid has no physical recycle content, but has credit-based recycle content (dashed lines), is provided. In the process and system depicted in FIG. 2, the r-ethylene, r-propylene, r-methanol, and r-CO are not directly fed to the carbonylation facility 22, nor is the r-syngas used to produce methanol.

Instead, recycle content credits from the recycle content streams shown in FIG. 2 (e.g., the r-ethylene, the r-propylene, the r-methanol, the r-syngas, the r-CO, the r-HC feed, and/or the r-CO₂) can be attributed to one or more streams in the production facility 10. For example, the recycle content credits from one or more of the above streams can be attributed to the ethylene, propylene, methanol, and/or CO fed into the carbonylation facility 22. As such, the r-ethylene, the r-propylene, the r-methanol, the r-CO and, when used, the r-syngas each act as a “source material” of recycle content credits and the ethylene, propylene, methanol, and/or CO fed into the carbonylation facility 22 each act as a “target material” to which the recycle content credits are attributed.

In an embodiment or in combination with any embodiment mentioned herein, the source material has physical recycle content and the target material has less than 100 percent physical recycle content. For example, the source material can have at least 10, at least 25, at least 50, at least 75, at least 90, at least 99, or 100 percent physical recycle content and/or the target material can have less than 100, less than 99, less than 90, less than 75, less than 50, less than 25, less than 10, or less than 1 percent physical recycle content.

The ability to attribute recycle content credits from a source material to a target material removes the co-location requirement for the facility making the source material (with physical recycle content) and the facility making the organic acid. This allows a chemical recycling facility/site in one location to process waste material into one or more recycle content source materials and then apply recycle content credits from those source materials to one or more target materials being processed in existing commercial facilities located remotely from the chemical recycling facility/site. Further, the use of recycle content credits allows different entities to produce the source material and the r-organic acid. This allows efficient use of existing commercial assets to produce r-organic acid.

In an embodiment or in combination with any embodiment mentioned herein, the source material, including any of the streams disclosed above, is made at a facility/site that is at least 0.1, at least 0.5, at least 1, at least 5, at least 10, at least 50, at least 100, at least 500, or at least 1,000 miles from the facility/site where the target material is used to make the organic acids.

The attributing of recycle content credits from the source material (e.g., the r-ethylene produced from the pyrolysis of waste plastic) to the target material (e.g., the ethylene fed to the carbonylation facility) can be accomplished by transferring recycle content credits directly from the source material to the target material. Alternatively, as shown in FIG. 2, recycle content credits can be applied from any of the waste plastic, the recycle content hydrocarbon feed (r-HC feed), the r-syngas, the r-CO, the r-methanol, the r-ethylene, the r-propylene, and/or the r-CO₂ to the organic acid via a recycle content inventory 24. The recycle content inventory 24 can be a digital inventory or database used to record and track recycle content for various materials at various sites over various time periods.

When a recycle content inventory 24 is used, recycle content credits from the source material having physical recycle content (e.g., the waste plastic, the r-HC feed, the r-ethylene, the r-propylene, the r-syngas, the r-CO, the r-methanol, and/or the r-CO₂ in FIG. 2) are booked into the recycle content inventory 24. The recycle content inventory 24 can also contain recycle content credits from other sources and from other time periods.

In an embodiment or in combination with any embodiment mentioned herein, recycle content credits in the recycle content inventory 24 can only be assigned to target materials having the same or similar composition as the source materials. For example, as shown in FIG. 2, recycle content credits booked into the recycle content inventory 24 from the r-ethylene from pyrolysis/cracking 12, 14 can be assigned to the ethylene fed to the carbonylation facility 22 because the two ethylene streams have the same or similar compositions. However, recycle content credits from r-ethylene could not be assigned to the methanol fed to the carbonylation facility 22 because the source and target materials would not be the same or similar.

In an embodiment or in combination with any embodiment mentioned herein, all or a portion of the recycle content credit can be applied to one or more target materials (e.g., methanol) upon receipt of one or more waste plastic containing materials at the facility. That is, the waste plastic (or recycle content hydrocarbon feed) need not be processed before applying the credit-based recycle content to the target material. Instead, receipt of the waste plastic (or waste-plastic containing material) at the facility can permit application of recycle content credit to one or more target materials. In most cases, however, such waste plastic will then be processed at the facility within 30, 60, or 90 days to produce one or more of the target materials. Once recycle content credits have been attributed to the target material (e.g., the ethylene, propylene, methanol, and/or CO in FIG. 2), the amount of the credit-based recycle content allocated to the organic acid is calculated by tracing the recycle content along the chemical pathway from the target material to the organic acid. The chemical pathway includes all chemical reactions and other processing steps (e.g., separations) between the target material and the organic acid, and a conversion factor can be associated with each step along the chemical pathway of the credit-based recycle content. The conversion factors account for the amount of the recycle content diverted or lost at each step along the chemical pathway. For example, the conversion factors can account for the conversion, yield, and/or selectivity of the chemical reactions along the chemical pathway.

As with the physical recycle content, the amount of credit-based recycle content applied to the r-organic acid can be determined using one of variety of methods, such as mass balance, for quantifying, tracking, and allocating recycle content among various materials in various processes. In certain embodiments, the method of quantifying, tracking, and allocating recycle content is overseen by a certification entity that confirms the accuracy of the method and provides certification for the application of recycle content to the r-organic acid.

In an embodiment or in combination with any embodiment mentioned herein, the r-organic acid can comprise credit-based recycle content from one or more of the source materials. For instance, at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent of the recycle content of the recycle content organic acid may be derived from credit-based recycle content. Additionally, or in the alternative, less than 99, less than 90, less than 80, less than 70, less than 60, less than 50, less than 40, less than 30, or less than 25 percent of the recycle content of the r-organic acid may be derived from credit-based recycle content. In certain embodiments, all of the recycle content of the r-organic acid is derived from credit-based recycle content.

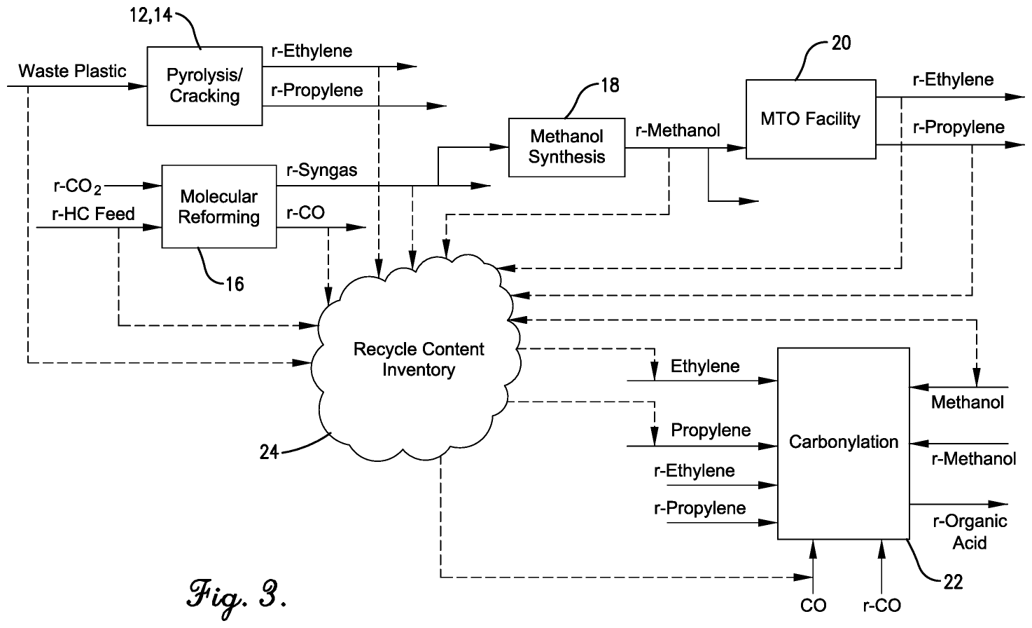

FIG. 3 illustrates several embodiments of an r-organic acid production process and system, wherein physical recycle content and credit-based recycle content are attributed to the r-organic acid. Any combination of physical (solid lines) and credit-based (dashed lines) recycle content shown in FIG. 3 can be used to form and/or can be attributed to the organic acid to thereby produce r-organic acid. For example, physical recycle content can be supplied by at least 1, at least 2, at least 3, at least 4, at least 5, or all of the sources shown in FIG. 3, including the r-ethylene, the r-propylene, the r-CO, the r-syngas, the r-HC feed, the r-CO₂, and the r-methanol, while the credit-based recycle content can be supplied by one or more of the other sources shown in FIG. 3.

In an embodiment or in combination with any embodiment mentioned herein, the r-organic acid can comprise at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 weight percent of total recycle content, derived from physical recycle content and/or credit-based recycle content. Additionally, or in the alternative, the r-organic acid can comprise less than 99, less than 90, less than 80, less than 70, less than 60, less than 50, less than 40, less than 30, or less than 25 weight percent of total recycle content, derived from physical recycle content and/or credit-based recycle content.

In an embodiment or in combination with any embodiment mentioned herein, the r-organic acid can comprise at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent physical recycle content and/or at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 99 percent credit-based recycle content. Additionally, or in the alternative, the r-organic acid can comprise less than 99, less than 90, less than 80, less than 70, less than 60, less than 50, less than 40, less than 30, or less than 25 percent physical recycle content and/or less than 99, less than 90, less than 80, less than 70, less than 60, less than 50, less than 40, less than 30, or less than 25 percent credit-based recycle content.

In an embodiment or in combination with any embodiment mentioned herein, the r-organic acid can include 10 to 60, 20 to 50, or 25 to 40 percent physical recycle content and 10 to 60, 20 to 50, or 25 to 40 percent credit-based recycle content. Alternatively, the r-organic acid can include less than 15, less than 10, or less than 5 percent physical recycle content (or credit-based recycle content) and at least 85, at least 90, or at least 95 percent credit based recycle content (or physical recycle content).

For example, in an embodiment or in combination with any embodiment mentioned herein, physical recycle content can be provided by r-ethylene fed to the carbonylation facility 22, while credit-based recycle content can be provided by methanol fed to the same process. In other embodiments, the physical recycle content can be provided by the r-methanol, while the ethylene can provide credit-based recycle content. Alternatively, both the methanol and ethylene can be the source of physical recycle content, or both can provide credit-based recycle content.

The r-organic acid produced by the process of FIG. 3 can have 25 to 90, 40 to 80, or 55 to 65 percent credit-based recycle content and less than 50, less than 25, less than 10, less than 5, or less than 1 percent physical recycle content. In certain embodiments, the r-organic acid can have 10 to 80, 20 to 75, or 25 to 70 percent credit-based recycle content from one or more of the r-syngas, the r-CO, the r-methanol, the r-ethylene, the r-propylene, the r-CO₂, and the r-HC feed, individually.

In an embodiment or in combination with any embodiment mentioned herein, the recycle content of the r-organic acid product can include both physical recycle content and credit-based recycle content. For example, the r-organic acid can have at least 10, at least 20, at least 30, at least 40, or at least 50 percent physical recycle content and at least 10, at least 20, at least 30, at least 40, or at least 50 percent credit-based recycle content.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from the waste plastic, derived from physical recycle content and/or credit-based recycle content. Additionally, or in the alternative, the r-organic acid, such as the r-propionic acid and/or the r-butyric acid, may comprise not more than 99, no more than 90, not more than 80, not more than 70, not more than 65, not more than 60, not more than 55, not more than 50, not more than 45, or not more than 40 percent recycle content from the waste plastic, derived from physical recycle content and/or credit-based recycle content.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from the r-syngas, derived from physical recycle content and/or credit-based recycle content. Additionally, or in the alternative, the r-organic acid, such as the r-propionic acid and/or the r-butyric acid, may comprise not more than 70, not more than 65, not more than 60, not more than 55, not more than 50, not more than 45, or not more than 40 percent recycle content from the r-syngas, derived from physical recycle content and/or credit-based recycle content.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from the r-CO, derived from physical recycle content and/or credit-based recycle content. Additionally, or in the alternative, the r-organic acid, such as the r-propionic acid and/or the r-butyric acid, may comprise not more than 70, not more than 65, not more than 60, not more than 55, not more than 50, not more than 45, or not more than 40 percent recycle content from the r-CO, derived from physical recycle content and/or credit-based recycle content.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from the r-C1-C6 alkanol (e.g., r-methanol), derived from physical recycle content and/or credit-based recycle content. Additionally, or in the alternative, the r-organic acid, such as the r-propionic acid and/or the r-butyric acid, may comprise not more than 70, not more than 65, not more than 60, not more than 55, not more than 50, not more than 45, or not more than 40 percent recycle content from the r-C1-C6 alkanol (e.g., r-methanol), derived from physical recycle content and/or credit-based recycle content.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from r-ethylene, derived from physical recycle content and/or credit-based recycle content. Additionally, or in the alternative, the r-organic acid, such as the r-propionic acid and/or the r-butyric acid, may comprise not more than 70, not more than 65, not more than 60, not more than 55, not more than 50, not more than 45, or not more than 40 percent recycle content from the r-ethylene, derived from physical recycle content and/or credit-based recycle content. This r-ethylene may include any or all of the r-ethylene streams from the pyrolysis facility 12, the cracking facility 14, and the MTO facility 20 described herein.

In an embodiment or in combination with any embodiment mentioned herein, the r-organic acid, such as the r-propionic acid and/or the r-butyric acid, may comprise at least 1, at least 2, at least 5, at least 10, at least 15, at least 20, at least 25, at least 30, or at least 35 percent recycle content from r-propylene, derived from physical recycle content and/or credit-based recycle content. Additionally, or in the alternative, the r-organic acid, such as the r-propionic acid and/or the r-butyric acid, may comprise not more than 70, not more than 65, not more than 60, not more than 55, not more than 50, not more than 45, or not more than 40 percent recycle content from the r-propylene, derived from physical recycle content and/or credit-based recycle content. This r-propylene may include any or all of the r-propylene streams from the pyrolysis facility 12, the cracking facility 14, and the MTO facility 20 described herein.

Turning again to FIG. 1, the pyrolysis facility 12, the cracking facility 14, the molecular reforming facility 16, the methanol catalytic synthesis facility 18, and/or the MTO facility 20 can be co-located with the carbonylation facility 22. Alternatively, in the embodiment shown in FIG. 2, the pyrolysis facility 12, the cracking facility 14, the molecular reforming facility 16, the methanol catalytic synthesis facility 18, and/or the MTO facility 20 can be remotely located from the carbonylation facility 22. In the embodiment shown in FIG. 3, the portions of the pyrolysis facility 12, the cracking facility 14, the molecular reforming facility 16, the methanol catalytic synthesis facility 18, and/or the MTO facility 20 providing physical recycle content to the carbonylation facility 22 may be co-located, while the portions of the pyrolysis facility 12, the cracking facility 14, the molecular reforming facility 16, the methanol catalytic synthesis facility 18, and/or the MTO facility 20 providing credit-based recycle content to the carbonylation facility 22 may be remotely located. When remotely located, the two facilities can be at least 0.5, at least 1, at least 5, at least 10, at least 100, at least 500, at least 1,000, or at least 10,000 miles from the other. When co-located, the two facilities can be within 10, within 5, within 2, within 1, within 0.5, or within 0.25 miles of one another. Furthermore, when remotely located, two or more of the facilities may be owned and/or operated by the same or by different commercial entities.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “carbonylation facility” refers to a facility that includes all equipment, lines, and controls necessary to carry out carbonylation of ethylene and/or propylene to produce an organic acid.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and CO) that are useful by themselves and/or are useful as feedstocks to another chemical production process(es).

As used herein, the term “co-located” refers to the characteristic of at least two objects being situated on a common physical site, and/or within 5, 1, 0.5, or 0.25 miles of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “credit-based recycle content,” “non-physical recycle content,” and “indirect recycle content” all refer to matter that is not physically traceable back to a waste material, but to which a recycle content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycle content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycle content based on the mass of the recycle content in various materials.

As used herein, the term “molecular reforming” refers to partial oxidation (POX) Gasification and steam reforming.

As used herein, the term “molecular reforming facility” refers to a facility that includes all equipment, lines, and controls necessary to carry out molecular reforming of waste plastic and feedstocks derived therefrom.

As used herein, the term “MTO facility” refers to a facility that includes all equipment, lines, and controls necessary to carry out the methanol-to-olefins process.

As used herein, the terms “partial oxidation (POX) gasification” or “POX gasification” refers to high temperature conversion of a carbon-containing feed into syngas, (carbon monoxide, hydrogen, and carbon dioxide), where the conversion is carried out in the presence of a less than stoichiometric amount of oxygen. The feed to POX gasification can include solids, liquids, and/or gases.

As used herein, the term “partial oxidation (POX) reaction” refers to all reactions occurring within a partial oxidation (POX) gasifier in the conversion of a carbon-containing feed into syngas, including but not limited to partial oxidation, water gas shift, water gas—primary reactions, Boudouard, oxidation, methanation, hydrogen reforming, steam reforming, and carbon dioxide reforming.

As used herein, the terms “physical recycle content” and “direct recycle content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “pyrolysis” refers to thermal decomposition of a feedstock of a biomass and/or a plastic material in solid or liquid form at elevated temperatures in an inert (i.e., substantially molecular oxygen free) atmosphere.

As used herein, the term “pyrolysis char” refers to a carbon-containing composition obtained from pyrolysis that is solid at 200° C. and 1 atm.

As used herein, the terms “pyrolysis gas” and “pygas” refer to a composition obtained from pyrolysis that is gaseous at 25° C. at 1 atm.

As used herein, the term “pyrolysis heavy waxes” refers to C20+ hydrocarbons obtained from pyrolysis that are not pyrolysis char, pyrolysis gas, or pyrolysis oil.

As used herein, the terms “pyrolysis oil” or “pyoil” refers to a composition obtained from pyrolysis that is liquid at 25° C. and 1 atm.

As used herein, the term “pyrolysis residue” refers to a composition obtained from pyrolysis that is not pyrolysis gas or pyrolysis oil and that comprises predominantly pyrolysis char and pyrolysis heavy waxes.

As used herein, the term “recycle content” or “r-content” refer to being or comprising a composition that is directly and/or indirectly derived from recycle material. Recycle content is used generically to refer to both physical recycle content and credit-based recycle content. Recycle content is also used as an adjective to describe material having physical recycle content and/or credit-based recycle content.

As used herein, the term “recycle content credit” refers to a non-physical measure of physical recycle content that can be directly or indirectly (i.e., via a digital inventory) attributed from a first material having physical recycle content to a second material having less than 100 percent physical recycle content.

As used herein, the terms “recycle content butyric acid” or “r-butyric acid” refer to being or comprising a butyric acid that is directly and/or indirectly derived from recycle material.

As used herein, the terms “recycle content CO” or “r-CO” refer to being or comprising CO that is directly and/or indirectly derived from recycle material.

As used herein, the terms “recycle content CO₂” or “r-CO₂” refer to being or comprising C02 that is directly and/or indirectly derived from recycle material.

As used herein, the terms “recycle content ethylene” or “r-ethylene” refer to being or comprising ethylene that is directly and/or indirectly derived from recycle material.

As used herein, the terms “recycle content methanol” or “r-methanol” refer to being or comprising methanol that is directly and/or indirectly derived from recycle material.

As used herein, the terms “recycle content organic acid” or “r-organic acid” refer to being or comprising an organic acid that is directly and/or indirectly derived from recycle material.

As used herein, the terms “recycle content propionic acid” or “r-propionic acid” refer to being or comprising a propionic acid that is directly and/or indirectly derived from recycle material.

As used herein, the terms “recycle content propylene” or “r-propylene” refer to being or comprising propylene that is directly and/or indirectly derived from recycle material.

As used herein, the terms “recycle content pyrolysis gas,” “r-pyrolysis gas,” or “r-pygas” refer to being or comprising a pyrolysis gas that is directly and/or indirectly derived from recycle material.

As used herein, the terms “recycle content pyrolysis oil,” “r-pyrolysis oil,” or “r-pyoil” refer to being or comprising a pyrolysis oil that is directly and/or indirectly derived from recycle material.

As used herein, the terms “recycle content syngas” or “r-syngas” refer to being or comprising a syngas that is directly and/or indirectly derived from recycle material.

As used herein, the term “total recycle content” refers to the cumulative amount of physical recycle content and credit-based recycle content from all sources.

As used herein, the term “waste material” refers to used, scrap, and/or discarded material.

As used herein, the terms “waste plastic” and “plastic waste” refer to used, scrap, and/or discarded plastic materials.

### Claims not Limited to Disclosed Embodiments

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

When a numerical sequence is indicated, it is to be understood that each number is modified the same as the first number or last number in the numerical sequence or in the sentence, e.g., each number is “at least,” or “up to” or “not more than” as the case may be; and each number is in an “or” relationship. For example, “at least 10, 20, 30, 40, 50, 75 wt. % . . . ” means the same as “at least 10 wt. %, or at least 20 wt. %, or at least 30 wt. %, or at least 40 wt. %, or at least 50 wt. %, or at least 75 wt. %,” etc.; and “not more than 90 wt. %, 85, 70, 60 . . . ” means the same as “not more than 90 wt. %, or not more than 85 wt. %, or not more than 70 wt. % . . . ” etc.; and “at least 1%, 2%, 3%, 4%, 5%, 6%, 7%, 8%, 9% or 10% by weight . . . ” means the same as “at least 1 wt. %, or at least 2 wt. %, or at least 3 wt. % . . . ” etc.; and “at least 5, 10, 15, 20 and/or not more than 99, 95, 90 weight percent” means the same as “at least 5 wt. %, or at least 10 wt. %, or at least 15 wt. % or at least 20 wt. % and/or not more than 99 wt. %, or not more than 95 wt. %, or not more than 90 weight percent . . . ” etc.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing a recycle content organic acid, the process comprising:
reacting ethylene and/or propylene with a C1-C6 alkanol, water, and carbon monoxide in the presence of a catalyst system to thereby produce a recycle content organic acid,
wherein at least a portion of the ethylene, the propylene, the carbon monoxide, or the C1-C6 alkanol comprise one or more of the following source materials—
(i) a recycle content ethylene,
(ii) a recycle content carbon monoxide,
(iii) a recycle content C1-C6 alkanol, and/or
(iv) a recycle content propylene.

2. The process according to claim 1, wherein the recycle content organic acid comprises recycle content propionic acid or recycle content butyric acid.

3. The process according to claim 1, wherein the recycle content organic acid comprises recycle content from the recycle content carbon monoxide.

4. The process according to claim 1, wherein the recycle content organic acid comprises recycle content from the recycle content C1-C6 alkanol.

5. The process according to claim 1, wherein the recycle content organic acid comprises recycle content from the recycle content ethylene.

6. The process according to claim 1, wherein the recycle content organic acid comprises recycle content from the recycle content propylene.

7. The process according to claim 1, wherein the recycle content ethylene and/or the recycle content propylene are formed via pyrolysis of a waste plastic, cracking of a pyrolysis effluent, a methanol-to-olefin process, or a combination thereof.

8. The process according to claim 1, wherein the reacting occurs at a temperature of 50 to 200° C., and at a pressure of at least 1,375 kPa.

9. A process for producing a recycle content organic acid, the process comprising:
(a) pyrolyzing a waste plastic to form a pyrolysis effluent comprising a first recycle content ethylene and/or a first recycle content propylene;
(b) optionally cracking at least a portion of the pyrolysis effluent to form a second recycle content ethylene and/or a second recycle content propylene;
(c) gasifying at least a portion of the pyrolysis effluent to form a recycle content syngas;
(d) optionally forming a recycle content C1-C6 alkanol with at least a portion of the recycle content syngas;
(e) optionally forming a recycle content carbon monoxide with at least a portion of the recycle content syngas;
(f) optionally forming a third recycle content ethylene and/or a third recycle content propylene with at least a portion of the recycle content C1-C6 alkanol; and
(g) reacting ethylene and/or propylene with a C1-C6 alkanol, water, and carbon monoxide in the presence of a catalyst system to thereby produce a recycle content organic acid, wherein the recycle content organic acid comprises recycle content from one or more of the following source materials—
(i) the waste plastic,
(ii) the first recycle content ethylene,
(iii) the first recycle content propylene,
(iv) the second recycle content ethylene,
(v) the second recycle content propylene,
(vi) the recycle content syngas,
(vii) the recycle content carbon monoxide,
(viii) the recycle content C1-C6 alkanol,
(ix) the third recycle content ethylene, and/or
(x) the third recycle content propylene.

10. The process according to claim 9, wherein the recycle content organic acid comprises recycle content propionic acid or recycle content butyric acid.

11. The process according to claim 10, wherein the recycle content organic acid comprises physical recycle content from one or more of the source materials.

12. The process according to claim 9, wherein the reacting is carried out at a carbonylation facility, wherein at least one of the source materials providing recycle content to the recycle content organic acid is produced in a co-located source facility that is located within 10, within 5, or within 2 miles from the carbonylation facility.

13. A process for producing a recycle content organic acid, the process comprising:
(a) treating a waste plastic to obtain, optionally through one or more intermediate processes, a recycle content ethylene, a recycle content propylene, a recycle content carbon monoxide, a recycle content methanol, or a combination thereof; and
(b) reacting ethylene and/or propylene with a C1-C6 alkanol, water, and carbon monoxide in the presence of a catalyst system to thereby produce a recycle content organic acid, wherein at least a portion of the ethylene, the propylene, the C1-C6 alkanol, or the carbon monoxide comprises the recycle content ethylene, the recycle content propylene, the recycle content methanol, or the recycle content carbon monoxide, respectively.

14. The process according to claim 13, wherein the recycle content organic acid comprises recycle content from the recycle content C1-C6 methanol.

15. The process according to claim 13, wherein the recycle content organic acid comprises recycle content from the recycle content ethylene.

16. The process according to claim 13, wherein the recycle content organic acid comprises recycle content from the recycle content propylene.

17. The process according to claim 13, wherein the intermediate processes comprise pyrolysis to produce a pyrolysis effluent comprising the recycle content ethylene and/or the recycle content propylene.

18. The process according to claim 13, wherein the intermediate processes comprise cracking recycle content pyrolysis oil and/or a recycle content pyrolysis gas to obtain the recycle content ethylene and/or the recycle content propylene.

19. The process according to claim 13, wherein the intermediate processes comprise feed a recycle content pyrolysis effluent through one or more process steps, wherein the process steps comprise a fluidized catalytic cracker.

20. The process according to claim 13, wherein the intermediate processes comprise gasifying the waste plastic to obtain the recycle content carbon monoxide.
